(19) United States
(12) Patent Application Publication
(10) Pub. No.: US 2021/0004617 A1
(43) Pub. Date: Jan. 7, 2021

(54) METHOD AND SYSTEM FOR MONITORING A STATE OF CONSCIOUSNESS OF AN OPERATOR IN AN AIRCRAFT COCKPIT

(71) Applicant: Airbus Operations (S.A.S.), Toulouse (FR)

(72) Inventors: Jonas Gouraud, Blagnac (FR); Romain Chaumillon, Toulouse (FR); Cecile Vollard-Derme, Toulouse (FR); Daniel Lewkowicz, Toulouse (FR)

(21) Appl. No.: 16/913,823

(22) Filed: Jun. 26, 2020

(30) Foreign Application Priority Data
Jul. 5, 2019 (FR) 1907512

(51) Int. Cl. G06K 9/00

(52) U.S. Cl. CPC G06K 9/00845; G06K 9/00288; G06K 9/00335

(57) ABSTRACT

A method and system for monitoring a state of consciousness of an operator in an aircraft cockpit includes at least one image acquisition module for acquiring images of the operator, a detection module for detecting movements of the body of the operator from the images, a determination module for determining a movement feature of the body of the operator during at least one time proportion of a predetermined time window, a transmission module for transmitting a signal representative of a state of loss of consciousness of the operator to a user device. The transmission module is implemented if the movement feature meets a predetermined condition. The state of consciousness of the operator is thus monitored automatically in order to supplement the monitoring by the flight crew members.

### CROSS-REFERENCE TO RELATED APPLICATION

This application claims the benefit of and priority to French patent application number 19 07512 filed on Jul. 5, 2019, the entire disclosure of which is incorporated by reference herein.

### TECHNICAL FIELD

The disclosure herein relates to monitoring a state of consciousness of an operator, in particular an aircraft pilot, in a cockpit of an aircraft.

### BACKGROUND

Monitoring the state of consciousness of an operator in his cockpit, for example a pilot on board an aircraft, is a vital action in order to detect a loss of consciousness, which may lead to the pilot being unable to perform his activities. The definition of a loss of consciousness may be as follows: loss of the ability to maintain awareness of self and environment combined with markedly reduced responsiveness to environmental sensory stimuli.

At present, the state of consciousness of an operator is monitored by human beings, in particular a copilot or a flight crew member. In order to supplement monitoring by the copilot, it would be desirable to provide an automated solution for detecting loss of consciousness.

### SUMMARY

The disclosure herein aims to overcome these drawbacks by proposing a method and a system for automatically monitoring the state of consciousness of an operator.

To this end, the disclosure herein relates to a method for monitoring a state of consciousness of at least one operator in an aircraft cockpit.

According to the disclosure herein, the method comprises at least one set of steps comprising at least the following steps, implemented automatically and iteratively:
an image acquisition step, implemented by an image acquisition module, consisting in or comprising acquiring images of the operator or operators,
a first detection step, implemented by a first detection module, consisting in or comprising detecting at least one first movement of the body of the operator or operators from the images,
a first determination step, implemented by a first determination module, consisting in or comprising determining a first movement feature of the body of the operator or operators during at least one first time proportion of a predetermined first time window, the first determination step being implemented if at least one first movement of the body of the operator or operators is detected during at least the first time proportion,
a first transmission step, implemented by a first transmission module, consisting in or comprising transmitting a signal representative of a state of loss of consciousness of the operator or operators to a user device, the first transmission step being implemented if the first movement feature meets a predetermined first condition.

Thus, by virtue of the method, the state of consciousness of the operator or operators is monitored automatically in order to supplement the monitoring by the flight crew members.

The method furthermore comprises:
a second detection step, implemented by a second detection module, consisting in or comprising detecting at least one second movement of the body of the operator or operators from the images, the second detection step being implemented:
   if no first movement of the body of the operator or operators is detected during the first time proportion, or
   if the first movement feature does not meet the predetermined first condition,
a second determination step, implemented by a second determination module, consisting in or comprising determining a second movement feature of the body of the operator or operators during at least one second time proportion of a second time window, the second determination step being implemented if at least one second movement of the body of the operator or operators is detected during at least the second time proportion,
a second transmission step, implemented by a second transmission module, consisting in or comprising transmitting a signal representative of a state of loss of consciousness of the operator or operators to the user device, the second transmission step being implemented if the second movement feature meets a predetermined second condition.

The method additionally comprises:
a third transmission step, implemented by a third transmission module, consisting in or comprising transmitting a signal representative of a possible state of malfunctioning of the movement detection to the user device, the third transmission step being implemented if no second movement of the body of the operator or operators is detected during the second time proportion.

According to a first embodiment, the first movement or movements of the body of the operator or operators correspond to at least one blink of at least one eye of the operator or operators, the first movement feature corresponding to a number of blinks of at least one eye, the first movement feature meeting the predetermined first condition if the number of blinks of at least one eye is less than a predetermined number of blinks of at least one eye and the second movement or movements of the body of the operator or operators corresponding to at least one head direction movement of the operator or operators, the second movement feature corresponding to a head direction movement speed, the second movement feature meeting the predetermined second condition if the head direction movement speed is less than a predetermined head direction movement speed.

According to a second embodiment, the first movement or movements of the body of the operator or operators correspond to at least one head direction movement of the operator or operators, the first movement feature corresponding to a head direction movement speed, the first movement feature meeting the predetermined first condition if the head direction movement speed is less than a predetermined head direction movement speed and the second movement or movements of the body of the operator or operators corresponding to at least one blink of at least one eye of the operator or operators, the second movement feature corresponding to a number of blinks of at least one eye, the second movement feature meeting the predetermined second condition if the number of blinks of at least one eye is less than a predetermined number of blinks of at least one eye.

According to a third embodiment, the first movement feature corresponds to a sequence of movements of at least one body part of the operator or operators, the first movement feature meeting the predetermined first condition if the sequence of movements is similar to a predetermined sequence of movements, the first movement feature not meeting the predetermined first condition if the sequence of movements is not similar to the predetermined sequence of movements.

According to a first variant, the method comprises:
a third determination step, implemented by a third determination module, consisting in or comprising determining a head orientation of the operator or operators from the images, the third determination step being implemented if the head direction movement speed is greater than or equal to the predetermined head direction movement speed,
a fourth transmission step, implemented by a fourth transmission module, consisting in or comprising transmitting a signal representative of a state of loss of consciousness of the operator to the user device, the second transmission step being implemented if the head orientation is within a predetermined orientation range during at least one third time proportion of the second time window.

According to a second variant, the method comprises:
a third detection step, implemented by a third detection module, consisting in or comprising detecting a shoulder movement of the operator or operators from the images, the third detection step being implemented:
   if the second movement feature does not meet the predetermined second condition, or
   if the head orientation is greater than or equal to the predetermined orientation during at least the third time proportion,
a fourth determination step, implemented by a fourth determination module, consisting in or comprising determining a shoulder movement speed of the operator or operators during at least the third time proportion, the fourth determination step being implemented if at least one shoulder movement of the operator or operators is detected during at least the third time proportion,
a fifth transmission step, implemented by a fifth transmission module, consisting in or comprising transmitting a signal representative of a state of loss of consciousness of the operator to the user device, the fifth transmission step being implemented if the shoulder movement speed is less than a predetermined shoulder movement speed.

According to a third variant, the method comprises an initialization step, implemented by an initialization module, the initialization step comprising at least one first detection substep, implemented by a first detection submodule, consisting in or comprising detecting an action of the operator or operators on at least one control device on board the aircraft, the set of steps being implemented if no action has been detected during a predetermined first period.

According to a fourth variant, the method comprises:
a fourth detection step, implemented by a fourth detection module, consisting in or comprising detecting a head direction movement of the operator or operators from the images, the fourth detection step being implemented if no movement of the second body part of the operator or operators has been detected,
a fifth determination step, implemented by a fifth determination module, consisting in or comprising determining a third head direction movement feature of the operator or operators during at least one fourth time proportion of the second time window, the fifth determination step being implemented if at least one head direction movement of the operator or operators is detected during at least the fourth time proportion,
a sixth transmission step, implemented by a sixth transmission module, consisting in or comprising transmitting a signal representative of a state of loss of consciousness of the operator to the user device, the sixth transmission step being implemented if the third head direction movement feature meets a predetermined third condition,
a sixth determination step, implemented by a sixth determination module, consisting in or comprising determining a head direction angle of the operator or operators with respect to an axis of the aircraft from the images, the sixth determination step being implemented if no head direction movement of the operator or operators has been detected,
a seventh transmission step, implemented by a seventh transmission module, consisting in or comprising transmitting a signal representative of a state of loss of consciousness of the operator to the user device, the seventh transmission step being implemented if the direction angle is less than a predetermined direction angle,
an eighth transmission step, implemented by an eighth transmission module, consisting in or comprising transmitting a signal representative of possible malfunctioning of the image acquisition module to the user device, the eighth transmission step being implemented if the direction angle is greater than or equal to the predetermined direction angle.

According to a first embodiment, the third movement feature corresponds to a head direction movement speed, the third movement feature meeting the predetermined third condition if the head direction movement speed is greater than a predetermined head direction movement speed, the third movement feature not meeting the predetermined third condition if the head direction movement speed is less than or equal to the predetermined head direction movement speed.

According to a second embodiment, the third movement feature corresponds to a sequence of head direction movements, the third movement feature meeting the predetermined third condition if the sequence of head direction movements is similar to a predetermined sequence of head direction movements, the third movement feature not meeting the predetermined third condition if the sequence of head direction movements is not similar to the predetermined sequence of head direction movements.

According to a fifth variant, the initialization step comprises a second detection substep, implemented by a second detection submodule, consisting in or comprising detecting a pressure exerted by the mass of the operator or operators on a seat on which they are likely to sit, the set of steps being implemented:
firstly if the pressure has been detected during a predetermined second period, or
secondly if an action of the operator or operators has been detected during the predetermined first period and if the pressure has been detected during the predetermined second period.

In this fifth variant, the method furthermore comprises:
a seventh determination step, implemented by a seventh determination module, consisting in or comprising determining a distribution of the pressure exerted by the mass of the operator or operators on the seat, the seventh determination step being implemented:
   if no second movement of the body of the operator or operators has been detected, or
   if the direction angle is greater than or equal to the predetermined direction angle,
a ninth transmission step, implemented by a ninth transmission module, consisting in or comprising transmitting a signal representative of a state of loss of consciousness of the operator to the user device, the ninth transmission step being implemented if the distribution of the pressure exerted by the mass of the operator or operators on the seat has a difference proportion, in comparison with a reference pressure distribution, less than a predetermined proportion,
a tenth transmission step, implemented by a tenth transmission module, consisting in or comprising transmitting a signal representative of possible malfunctioning of the image acquisition module to the user device, the tenth transmission step being implemented if the distribution of the pressure exerted by the mass of the operator or operators on the seat has a difference proportion, in comparison with a reference pressure distribution, greater than or equal to the predetermined proportion.

According to a sixth variant, the method comprises:
a fifth detection step, implemented by a fifth detection module, consisting in or comprising detecting a heartbeat of the operator or operators, the fifth detection step being implemented:
   if no second movement of the body of the operator or operators has been detected, or
   if the distribution of the pressure exerted by the mass of the operator or operators on the seat has a difference proportion, in comparison with the reference pressure distribution, greater than or equal to the predetermined proportion,
an eighth determination step, implemented by an eighth determination module, consisting in or comprising determining a heart rate, the eighth determination step being implemented if at least one heartbeat of the operator or operators is detected,
an eleventh transmission step, implemented by an eleventh transmission module, consisting in or comprising transmitting a signal representative of a state of loss of consciousness of the operator or operators to the user device, the eleventh transmission step being implemented if the determined heart rate is not within a predetermined heart rate range,
a twelfth transmission step, implemented by a twelfth transmission module, consisting in or comprising transmitting a signal representative of possible malfunctioning of the image acquisition module to the user device, the twelfth transmission step being implemented if the determined heart rate is within the predetermined heart rate range.

The disclosure herein also relates to a system for monitoring a state of consciousness of at least one operator in an aircraft cockpit.

According to the disclosure herein, the system comprises at least one set of modules comprising at least:
an image acquisition module configured so as to acquire images of the operator,
a first detection module configured so as to detect at least one first movement of the body of the operator or operators from the images,
a first determination module configured so as to determine a first movement feature of the body of the operator or operators during at least one first time proportion of a predetermined first time window, the first determination module being implemented if at least one first movement of the body of the operator or operators is detected during the first time proportion,
a first transmission module configured so as to transmit a signal representative of a state of loss of consciousness of the operator to a user device, the first transmission module being implemented if the first movement feature meets a predetermined first condition.

The system additionally comprises:
a second detection module configured so as to detect at least one second movement of the body of the operator or operators from the images, the second detection module being implemented:
   if no first movement of the body of the operator or operators is detected during the first time proportion, or
   if the first movement feature does not meet the predetermined first condition,
a second determination module configured so as to determine a second movement feature of the body of the operator or operators during at least one second time proportion of a second time window, the second determination module being implemented if at least one second movement of the body of the operator or operators is detected during at least the second time proportion,
a second transmission module configured so as to transmit a signal representative of a state of loss of consciousness of the operator to the user device, the second transmission module being implemented if the second movement feature meets a predetermined second condition.

The system furthermore comprises:
a third transmission module configured so as to transmit a signal representative of a possible state of malfunctioning of the movement detection to the user device, the third transmission module being implemented if no second movement of the body of the operator or operators is detected during at least the second time proportion.

The disclosure herein also relates to an aircraft, in particular a transport plane, comprising a system for monitoring a state of consciousness of at least one operator in an aircraft cockpit, as described above.

### BRIEF DESCRIPTION OF THE FIGURES

The disclosure herein, with its features and advantages, will become more clearly apparent upon reading the description provided with reference to the appended drawings, in which:

FIG. 1 schematically shows the monitoring system.

FIG. 2 shows an aircraft incorporating the monitoring system.

FIG. 3 schematically shows a combination of a first variant and a second variant of the monitoring method.

FIG. 4 schematically shows a third variant of the monitoring method.

FIG. 5 schematically shows a fourth variant of the monitoring method.

FIG. 6 schematically shows a fifth variant of the monitoring method.

FIG. 7 schematically shows a sixth variant of the monitoring method.

FIG. 8 shows a variant that is a combination of the variants of the monitoring method.

### DETAILED DESCRIPTION

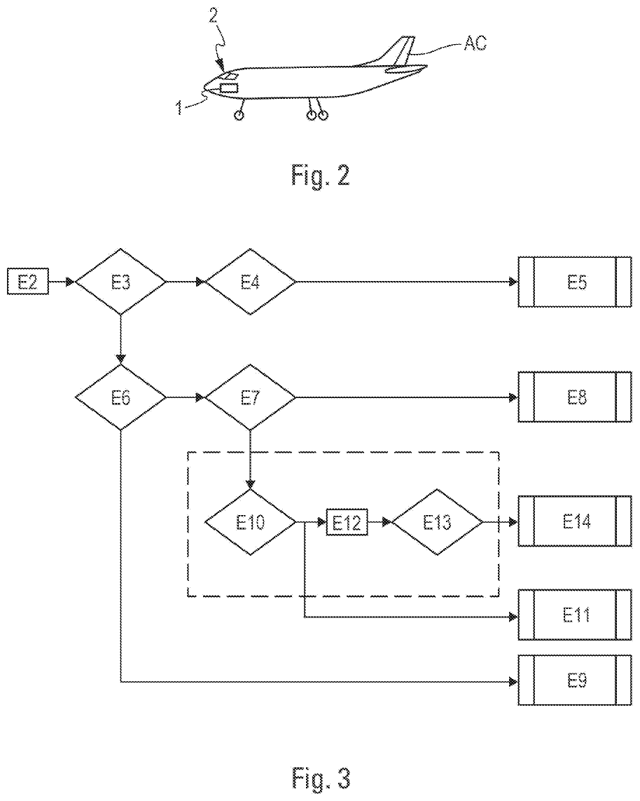

The disclosure herein relates to a system 1 for monitoring a state of consciousness of an operator in an aircraft AC cockpit 2 (FIG. 2). The system is called “monitoring system” in the remainder of the description.

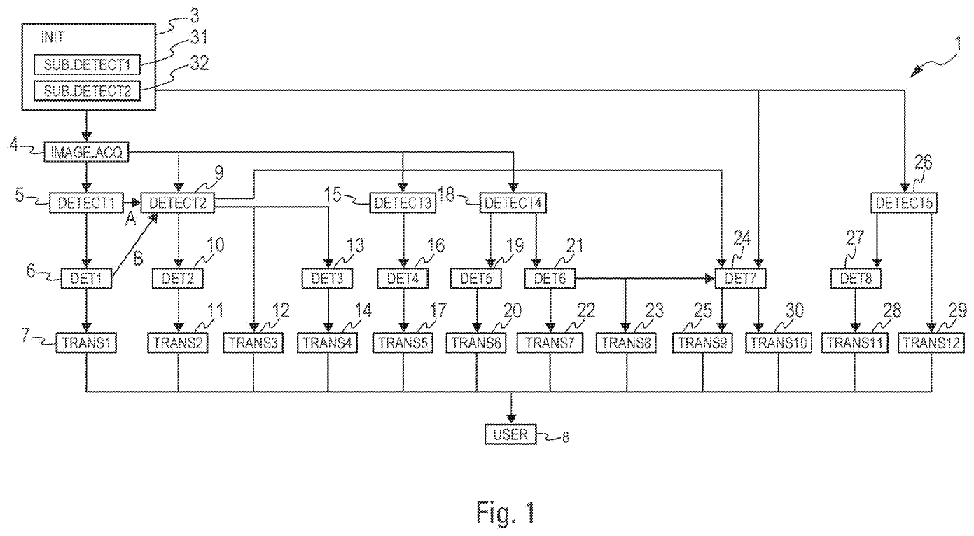

One embodiment of the monitoring system 1 is shown in FIG. 1.

The monitoring system comprises an initialization module INIT (INIT for initialization module) 3 configured so as to initialize the monitoring system 1, The initialization module 3 makes it possible to implement the monitoring system 1 under given conditions. For example, the given conditions may be the presence of an operator in the cockpit 2. Some embodiments of the initialization module 3 are presented below in the description.

The monitoring system 1 furthermore comprises:
an image acquisition module IMAGE_ACQ (IMAGE_ACQ for “image acquisition module”) 4 configured so as to acquire images of at least one operator,
a detection module DETECT1 (DETECT for “detection module”) 5 configured so as to detect at least one first movement of the body of the operator or operators from the images,
a determination module DET1 (DET for “determination module”) 6 configured so as to determine a first movement feature of the body of the operator or operators during at least one first time proportion of a predetermined first time window,
a transmission module TRANS1 (TRANS for “transmission module”) 7 configured so as to transmit a signal representative of a state of loss of consciousness of the operator or operators to a user device USER (USER for “user device”) 8.

The determination module 6 is configured so as to be implemented if at least one first movement of the body of the operator or operators is detected by the detection module 5 during at least the first time proportion.

The detection by the detection module 5 may be unavailable at certain times during the time window and available at other times during the time window. For example, the detection may be unavailable at times when the operator turns and his face is no longer visible to the image acquisition module 4, at times when there is an obstacle (book or other objects) between the face of the operator and the image acquisition module 4, or else at times when the image acquisition module 4 has an intermittent technical problem. A time proportion (or percentage) of a time window then corresponds to the ratio between the sum of the times at which detection is available during the time window, on the one hand, and the time window, on the other hand. This definition of a time proportion is the same in the remainder of the description.

The movement or movements of the body of the operator or operators may be one or more movements of at least one body part of the operator or operators. The body part or parts may correspond to at least one organ of the operator or operators that is able to be identified and/or monitored by the image acquisition module 4 and that is likely to move in a known way.

The transmission module 7 is configured so as to be implemented if the first movement feature determined by the determination module 6 meets a predetermined first condition.

The image acquisition module may correspond to a camera or may comprise a camera.

The monitoring system may furthermore comprise a detection module DETECT2 9 configured so as to detect at least one second movement of the body of the operator or operators from the images.

According to a first embodiment of the detection module 9, the detection module 9 is configured so as to be implemented if no first movement of the body of the operator or operators is detected by the detection module 5 during the first time proportion. In FIG. 1, a link A links the detection module 5 to the detection module 9.

According to a second embodiment of the detection module 9, the detection module 9 is configured so as to be implemented if the first movement feature determined by the determination module 6 meets the predetermined first condition. In FIG. 1, a link B links the determination module 6 to the detection module 9.

The monitoring system 1 may also comprise a determination module DET2 10 configured so as to determine a second movement feature of the body of the operator or operators during at least one second time proportion of a second time window.

The determination module 10 is configured so as to be implemented if at least one second movement of the body of the operator is detected by the detection module 9 during at least the second time proportion.

The monitoring system 1 may comprise a transmission module TRANS2 11 configured so as to transmit a signal representative of a state of loss of consciousness of the operator to the user device. The transmission module 11 is implemented if the second movement feature meets a predetermined second condition.

Advantageously, the monitoring system 1 comprises a transmission module TRANS3 12 configured so as to transmit a signal representative of a state of possible malfunctioning of the movement detection to the user device 8. Thus, if the detection modules 5 and 9 do not detect any first movement or possibly any second movement of the body of the operator or operators, the signal is transmitted to the user device 8 in order to indicate possible malfunctioning of the detection modules 5 and/or 9.

The transmission module 12 is implemented if no second movement of the body of the operator or operators is detected during at least the second time proportion.

According to a first embodiment, the first movement or movements of the body of the operator or operators correspond to at least one blink of the eye or eyes of the operator or operators and the second movement of movements of the body of the operator or operators correspond to a head direction movement of the operator or operators.

The first movement feature corresponds to a number of blinks of at least one eye. The first movement feature meets the predetermined first condition if the number of blinks of at least one eye is less than a predetermined number of blinks of at least one eye. The first movement feature does not meet the predetermined first condition if the number of blinks of at least one eye is greater than or equal to the predetermined number of blinks of at least one eye.

The second movement feature corresponds to a head direction movement speed. The second movement feature meets the predetermined second condition if the head direction movement speed is less than a predetermined head direction movement speed. The second movement feature does not meet the predetermined second condition if the head direction movement speed is greater than or equal to the predetermined head direction movement speed.

According to a second embodiment, the first movement or movements of the body of the operator or operators correspond to at least one head direction movement of the operator or operators and the second movement of movements of the body of the operator or operators correspond to at least one blink of the eye or eyes of the operator or operators.

The first movement feature corresponds to a head direction movement speed. The first movement feature meets the predetermined first condition if the head direction movement speed is less than a predetermined head direction movement speed. The first movement feature does not meet the predetermined first condition if the head direction movement speed is greater than or equal to the predetermined head direction movement speed.

The second movement feature corresponds to a number of blinks of at least one eye. The second movement feature meets the predetermined second condition if the number of blinks of at least one eye is less than a predetermined number of blinks of at least one eye. The second movement feature does not meet the predetermined second condition if the number of blinks of at least one eye is greater than or equal to the predetermined number of blinks of at least one eye.

Without limitation for the first embodiment and the second embodiment, in the case of blinks of the eye or eyes, the predetermined number of movements is between 0.5 and 5 blinks per minute and the time proportion is between 20% and 80% of the time window. In the case of head direction movements, the predetermined head direction movement speed is between 0 m/s and 10 m/s and the time proportion is between 20% and 80% of the time window.

According to a third embodiment, the first movement feature corresponds to a sequence (or pattern) of movements of at least one body part of the operator or operators. The first movement feature meets the predetermined first condition if the sequence of movements is similar to a predetermined sequence of movements. The first movement feature does not meet the predetermined first condition if the sequence of movements is not similar to the predetermined sequence of movements.

A sequence of movements may correspond to a set of successive or simultaneous or both successive and simultaneous movements of one or more body parts of the operator or operators. The movements may be at least blinks of at least one eye, head movements or shoulder movements. According to one non-limiting example, a sequence of movements corresponds to head movements followed by one or more eye blinks.

Without limitation, the similarity between a detected sequence and a predetermined sequence may be determined through movement recognition by way of image processing. The detected sequence is similar to the predetermined sequence when the detected sequence is close to the predetermined sequence, within a confidence interval.

According to a first variant embodiment, the monitoring system 1 comprises:
a determination module DET3 13 configured so as to determine a head orientation of the operator or operators from the images acquired by the image acquisition module 4,
a transmission module TRANS4 14 configured so as to transmit a signal representative of a state of loss of consciousness of the operator or operators to the user device 8.

The determination module 13 is configured so as to be implemented if the head direction speed determined by the determination module 10 is greater than or equal to the predetermined head direction movement speed.

The transmission module 14 is configured so as to be implemented if the head orientation determined by the determination module 13 is within a predetermined orientation range during at least one third time proportion of the second time window.

Without limitation, in the case of a head orientation in terms of pitch (with respect to the aircraft AC), the lower limit of the orientation range is between −60° and −10° and the upper limit is between 10° and 90°.

Without limitation, in the case of a head orientation in terms of roll (with respect to the aircraft AC), the lower limit is between −90° and −10° and the upper limit is between 10° and 90°.

According to a second variant, the monitoring system 1 comprises a detection module DETECT3 15 configured so as to detect a shoulder movement of the operator or operators from the images acquired by the image acquisition module 4.

According to a first embodiment of the detection module 15, the detection module 15 is configured so as to be implemented if the second movement feature determined by the determination module 10 does not meet the predetermined second condition.

According to a second embodiment of the detection module 15, the detection module 15 is configured so as to be implemented if the head orientation determined by the determination module 13 is greater than or equal to the predetermined orientation during at least the third time proportion.

In this second variant, the monitoring system 1 furthermore comprises:
a determination module DET4 16 configured so as to determine a shoulder movement speed of the operator or operators during at least the third time proportion,
a transmission module TRANS5 17 configured so as to transmit a signal representative of a state of loss of consciousness of the operator to the user device 8.

The determination module 16 is implemented if at least one shoulder movement of the operator is detected by the detection module 15 during at least the third time proportion.

The transmission module 17 is configured so as to be implemented if the shoulder movement speed determined by the determination module 16 is less than a predetermined shoulder movement speed.

Without limitation, the predetermined shoulder movement speed is between 0 m/s and 10 m/s.

According to a third variant, the initialization module furthermore comprises a detection submodule SUB_DETECT1 31 configured so as to detect an action of the operator or operators on at least one control device on board the aircraft AC. The modules of the monitoring system 1 may be implemented if no action has been detected during a predetermined first period.

The on-board control device may correspond to a human-machine interface (HMI).

The modules of the monitoring system 1 may preferably be implemented if no action has been detected during a predetermined first period on at least two separate control devices. This makes it possible to avoid detecting involuntary actions of the operator or operators.

According to a fourth variant, the monitoring system 1 furthermore comprises:
a detection module DETECT4 18 configured so as to detect a head direction movement of the operator or operators. The detection module 18 may correspond to an inertial unit mounted fixedly on the head of the operator or operators. For example, the inertial unit is mounted on a headset able to be worn by the operator or operators,
a determination module SETS 19 configured so as to determine a third head direction movement feature of the operator or operators during at least one fourth time proportion smaller than the second time window,
a transmission module TRANS6 20 configured so as to transmit a signal representative of a state of loss of consciousness of the operator or operators to the user device 8,
a determination module DET6 21 configured so as to determine a head direction angle of the operator or operators with respect to an axis of the aircraft AC from the images acquired by the image acquisition module 4. The axis of the aircraft AC may correspond to at least one or the other of the axes comprising the roll axis, the pitch axis and the yaw axis,
a transmission module TRANS7 22 configured so as to transmit a signal representative of a state of loss of consciousness of the operator to the user device 8,
a transmission module TRANS8 23 configured so as to transmit a signal representative of possible malfunctioning of the image acquisition module 4 to the user device 8.

The detection module 18 is configured so as to be implemented if no movement of the second body part of the operator or operators has been detected by the detection module 9 during at least the fourth time proportion. One example of not detecting movements of the second body part of the operator or operators may correspond to the case where the operator or operators turn(s) or lean(s) forward in order to speak to another operator or to access a particular area of the cockpit 2.

The determination module 19 is configured so as to be implemented if at least one head direction movement of the operator or operators is detected by the detection module 18 during at least the fourth time proportion.

The transmission module 20 is configured so as to be implemented if the third head direction movement feature meets a predetermined third condition.

According to a first embodiment, the third movement feature corresponds to a head direction movement speed. The third movement feature meets the predetermined third condition if the head direction movement speed is greater than a predetermined head direction movement speed. The third movement feature does not meet the predetermined third condition if the head direction movement speed is less than or equal to the predetermined head direction movement speed.

According to a second embodiment, the third movement feature corresponds to a sequence of head direction movements. The third movement feature meets the predetermined third condition if the sequence of head direction movements is similar to a predetermined sequence of head direction movements. The third movement feature does not meet the predetermined third condition if the sequence of head direction movements is not similar to the predetermined sequence of head direction movements.

The determination module 21 is configured so as to be implemented if no head direction movement of the operator or operators has been detected by the detection module 18.

The transmission module 22 is configured so as to be implemented if the direction angle determined by the determination module 21 is less than a predetermined direction angle.

The transmission module 23 is configured so as to be implemented if the direction angle determined by the determination module 21 is greater than or equal to the predetermined direction angle.

According to a fifth variant, the initialization module comprises a detection submodule SUB_DETECT2 32 configured so as to detect a pressure exerted by the mass of the operator or operators on at least one seat on which the operator or operators are likely to sit. The modules of the monitoring system 1 may be implemented if at least one operator is sitting on the seat.

The seat may be a pilot's seat.

The detection submodule 32 may correspond to a pressure mat integrated into the seat.

The monitoring system 1 may thus be initialized when at least one operator is seated.

According to a first embodiment of the detection submodule 32, the monitoring system 1 is initialized if the pressure has been detected by the detection submodule 32 during a predetermined second period.

According to a second embodiment of the detection submodule 32, the monitoring system 1 is initialized if an action of the operator has been detected by the detection submodule 31 during the predetermined first period and if the pressure has been detected by the detection submodule 32 during the predetermined second period.

In this fifth variant, the monitoring system 1 furthermore comprises:
a determination module DET7 24 configured so as to determine a distribution of the pressure exerted by the mass of the operator or operators on the seat,
a transmission module TRANS9 25 configured so as to transmit a signal representative of a state of loss of consciousness of the operator or operators to the user device 8.

According to a first embodiment of the determination module 24, the determination module 24 is configured so as to be implemented if no second movement of the body of the operator has been detected by the detection module 9. If the determination module 24 corresponds to the pressure mat, the determination module 24 may monitor a difference in mass distribution between the various parts of the body of the pressure mat.

According to a second embodiment of the determination module 24, the determination module 24 is configured so as to be implemented if the direction angle determined by the determination module 21 is greater than or equal to the predetermined direction angle.

The transmission module 25 is configured so as to be implemented if the distribution of the pressure exerted by the mass of the operator or operators on the seat has a difference proportion, in comparison with a reference pressure distribution, less than a predetermined proportion. For example, in the case of a pressure difference between two parts of the pressure mat that is greater than a predetermined threshold, it is considered that the operator or operators is or are leaning too far to one side due to a loss of consciousness.

Without limitation, the predetermined proportion is between 0% and 50%.

According to a sixth variant, the monitoring system 1 furthermore comprises a detection module DETECT5 26 configured so as to detect a heartbeat of the operator or operators.

According to a first embodiment of the detection module 26, the detection module 26 is implemented if no second movement of the body of the operator or operators has been detected by the detection module 9.

According to a second embodiment of the detection module 26, the detection module 26 is implemented if the distribution of the pressure exerted by the mass of the operator or operators on the seat determined by the determination module 24 has a difference proportion, in comparison with the reference pressure distribution, greater than or equal to the predetermined proportion.

In this sixth variant, the monitoring system 1 also comprises:
a determination module DET8 27 configured so as to determine a heart rate from the heartbeats detected by the detection module 26,
a transmission module TRANS11 28 configured so as to transmit a signal representative of a state of loss of consciousness of the operator or operators to the user device 8,
a transmission module TRANS12 29 configured so as to transmit a signal representative of possible malfunctioning of the image acquisition module 4 to the user device 8.

The determination module 27 is configured so as to be implemented if at least one heartbeat of the operator or operators is detected by the detection module 26.

The transmission module 28 is configured so as to be implemented if the heart rate determined by the determination module 27 is not within a predetermined heart rate range.

Without limitation, the lower limit of the heart rate range is between 0 beats per minute and 60 beats per minute, and the upper limit of the heart rate range is between 140 beats per minute and 220 beats per minute.

The transmission module 29 is configured so as to be implemented if the heart rate determined by the determination module 27 is within the predetermined heart rate range.

The variants of the method may be combined as shown in FIG. 1.

The disclosure herein also relates to a method for monitoring a state of consciousness of at least one operator in an aircraft AC cockpit 2 (FIGS. 3 to 8).

As shown in FIGS. 3 through 8, the method comprises at least one set of steps comprising at least the following steps, implemented automatically and iteratively:
an image acquisition step E2, implemented by the image acquisition module 4, consisting in or comprising acquiring images of the operator or operators,
a detection step E3, implemented by the detection module 5, consisting in or comprising detecting at least one first movement of the body of the operator or operators from the images,
a determination step E4, implemented by the determination module 6, consisting in or comprising determining the first movement feature of the body of the operator or operators during at least the first time proportion of the predetermined first time window, the determination step E4 being implemented if at least one first movement of the body of the operator or operators is detected during at least the first time proportion,
a transmission step E5, implemented by the transmission module 7, consisting in or comprising transmitting the signal representative of a state of loss of consciousness of the operator or operators to the user device 8, the transmission step E5 being implemented if the first movement feature meets a predetermined first condition.

The method may also comprise:
a detection step E6, implemented by the detection module 9, consisting in or comprising detecting at least one second movement of the body of the operator or operators from the images, the detection step E6 being implemented:
   if no first movement of the body of the operator or operators is detected during at least the first time proportion, or
   if the first movement feature meets the predetermined first condition,
a determination step E7, implemented by the determination module 10, consisting in or comprising determining the second movement feature of the body of the operator or operators during at least the second time proportion of the second time window, the determination step E7 being implemented if at least one second movement of the body of the operator or operators is detected during at least the second time proportion,
a transmission step E8, implemented by the transmission module 11, consisting in or comprising transmitting the signal representative of a state of loss of consciousness of the operator or operators to the user device 8, the transmission step E8 being implemented if the second movement feature meets a predetermined second condition.

The method may furthermore comprise a transmission step E9, implemented by the transmission module 12, consisting in or comprising transmitting the signal representative of a possible state of malfunctioning of the movement detection to the user device 8, the transmission step E9 being implemented if no second movement of the body of the operator or operators is detected during at least the second time proportion.

According to the first variant, the method may comprise:
a determination step E10, implemented by the determination module 13, consisting in or comprising determining the head orientation of the operator or operators from the images, the determination step E10 being implemented if the head direction movement speed is greater than or equal to the predetermined head direction movement speed,
a transmission step E11, implemented by the transmission module 14, consisting in or comprising transmitting the signal representative of a state of loss of consciousness of the operator to the user device 8, the transmission step E11 being implemented if the head orientation is within the predetermined orientation range during at least the third time proportion of the second time window.

According to the second variant, the method may comprise:
a detection step E12, implemented by the detection module 15, consisting in or comprising detecting a shoulder movement of the operator or operators from the images, the detection step E12 being implemented:
   if the second movement feature does not meet the predetermined second condition, or
   if the head orientation is greater than or equal to the predetermined orientation during at least the third time proportion,
a determination step E13, implemented by the determination module 16, consisting in or comprising determining the shoulder movement speed of the operator or operators during at least the third time proportion, the determination step E13 being implemented if at least one shoulder movement of the operator or operators is detected during at least the third time proportion,
a transmission step E14, implemented by the transmission module 17, consisting in or comprising transmitting the signal representative of a state of loss of consciousness of the operator to the user device 8, the transmission step E14 being implemented if the shoulder movement speed is less than the predetermined shoulder movement speed.

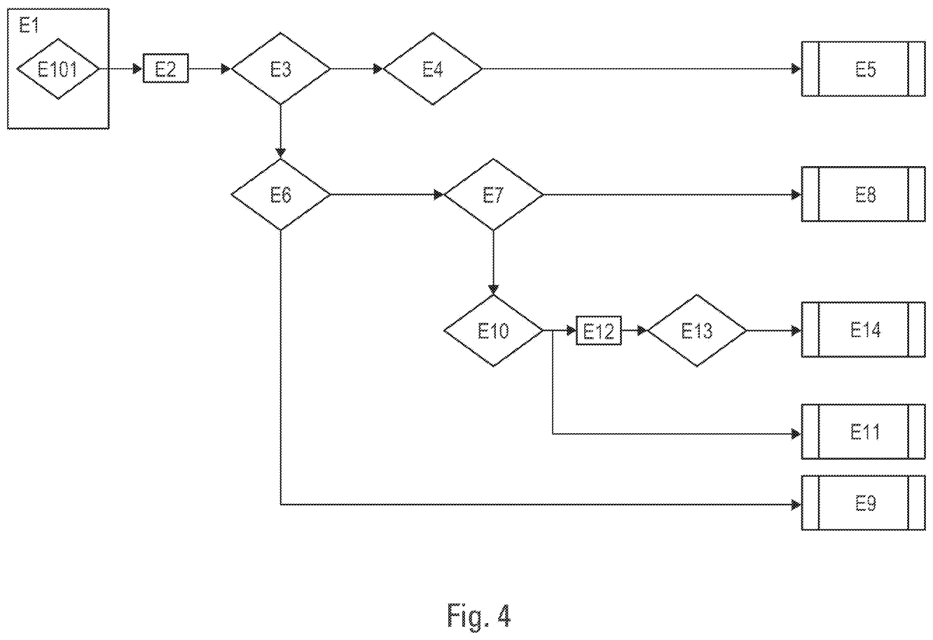

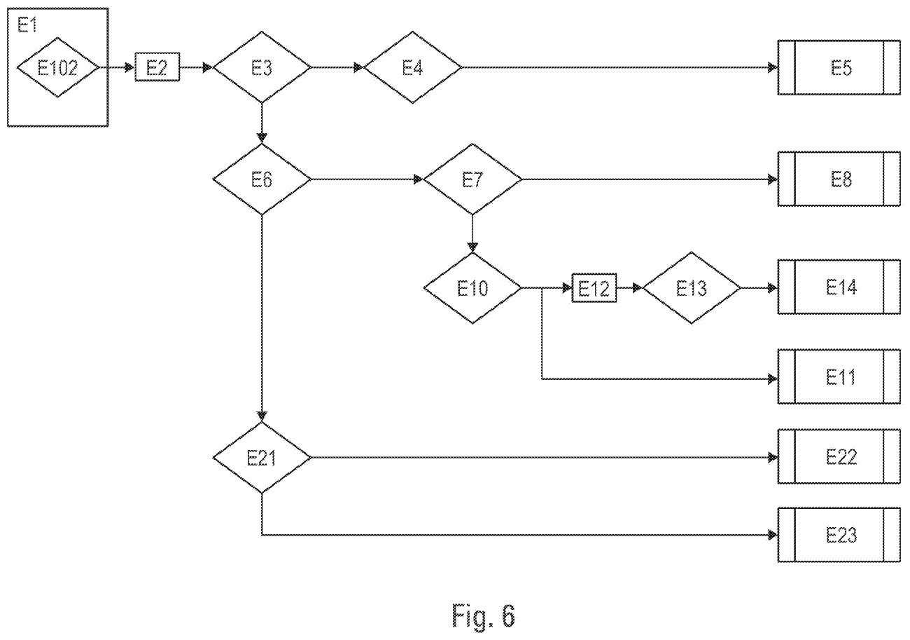

As shown in FIGS. 4 and 6, the method may comprise an initialization step E1, implemented by the initialization module 3.

According to the third variant shown in FIG. 4, the initialization step E1 comprises at least one detection substep E101, implemented by the detection submodule 31, consisting in or comprising detecting an action of the operator or operators on at least one control device on board the aircraft AC, the set of steps being implemented if no action has been detected during the predetermined first period.

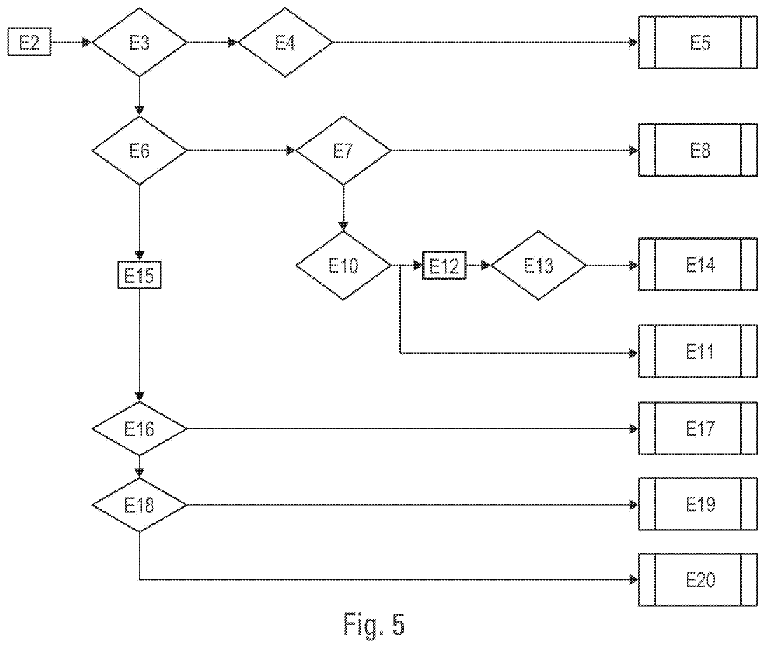

According to the fourth variant shown in FIG. 5, the method may comprise:
a detection step E15, implemented by the detection module 18, consisting in or comprising detecting the head direction movement of the operator or operators, the detection step E15 being implemented if no movement of the second body part of the operator or operators has been detected,
a determination step E16, implemented by the determination module 19, consisting in or comprising determining the third head direction movement feature of the operator or operators during at least the fourth time proportion of the second time window, the determination step E16 being implemented if at least one head direction movement of the operator or operators is detected during at least the fourth time proportion,
a transmission step E17, implemented by the transmission module 20, consisting in or comprising transmitting a signal representative of a state of loss of consciousness of the operator to the user device 8, the transmission step E17 being implemented if the third head direction movement feature meets the predetermined third condition,
a determination step E18, implemented by the determination module 21, consisting in or comprising determining the head direction angle of the operator or operators with respect to an axis of the aircraft AC from the images, the determination step E18 being implemented if no head direction movement of the operator or operators has been detected,
a transmission step E19, implemented by the transmission module 22, consisting in or comprising transmitting the signal representative of a state of loss of consciousness of the operator to the user device 8, the transmission step E19 being implemented if the direction angle is less than the predetermined direction angle,
a transmission step E20, implemented by the transmission module 23, consisting in or comprising transmitting the signal representative of possible malfunctioning of the image acquisition module 4 to the user device 8, the transmission step E20 being implemented if the direction angle is greater than or equal to the predetermined direction angle.

According to the fifth variant shown in FIG. 6, the initialization step E1 may comprise a detection substep E102, implemented by the detection submodule 32, consisting in or comprising detecting the pressure exerted by the mass of the operator or operators on the seat on which he or they are likely to sit, the set of steps being implemented:
firstly if the pressure has been detected during a predetermined second period, or
secondly if an action of the operator or operators has been detected during the predetermined first period and if the pressure has been detected during the predetermined second period.

In this case, the method furthermore comprises:
a determination step E21, implemented by the determination module 24, consisting in or comprising determining the distribution of the pressure exerted by the mass of the operator or operators on the seat, the determination step E21 being implemented:
   if no second movement of the body of the operator or operators has been detected, or
   if the direction angle is greater than or equal to the predetermined direction angle,
a transmission step E22, implemented by the transmission module 25, consisting in or comprising transmitting the signal representative of a state of loss of consciousness of the operator to the user device 8, the transmission step E22 being implemented if the distribution of the pressure exerted by the mass of the operator or operators on the seat has a difference proportion, in comparison with the reference pressure distribution, less than the predetermined proportion,
a transmission step E23, implemented by the transmission module 30, consisting in or comprising transmitting the signal representative of possible malfunctioning of the image acquisition module 4 to the user device 8, the transmission step E23 being implemented if the distribution of the pressure exerted by the mass of the operator or operators on the seat has a difference proportion, in comparison with the reference pressure distribution, greater than or equal to the predetermined proportion.

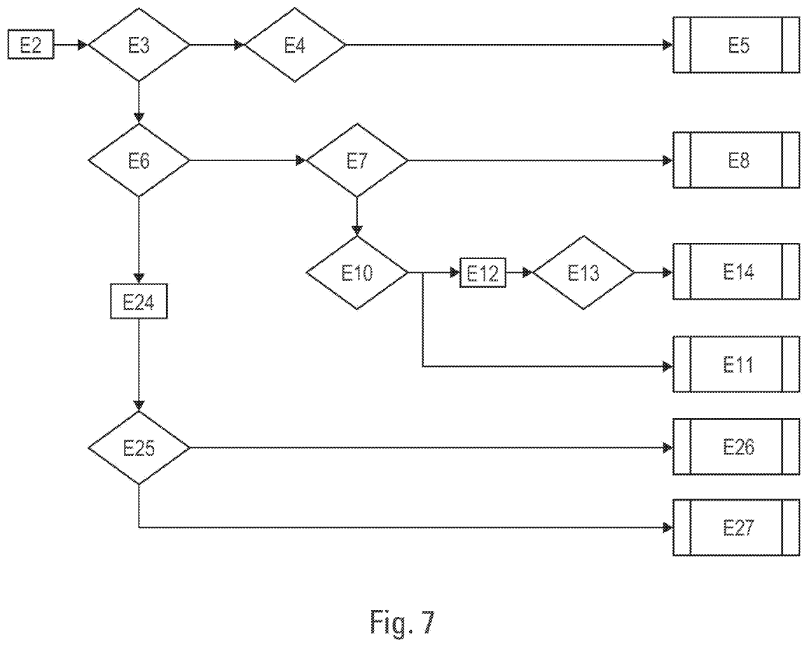

According to the sixth variant shown in FIG. 7, the method may comprise:
a detection step E24, implemented by the detection module 26, consisting in or comprising detecting the heartbeat of the operator or operators, the detection step E24 being implemented:
   if no second movement of the body of the operator or operators has been detected, or
   if the distribution of the pressure exerted by the mass of the operator or operators on the seat has a difference proportion, in comparison with the reference pressure distribution, greater than or equal to the predetermined proportion,
a determination step E25, implemented by the determination module 27, consisting in or comprising determining the heart rate, the determination step E25 being implemented if at least one heartbeat of the operator or operators is detected,
a transmission step E26, implemented by the transmission module 28, consisting in or comprising transmitting the signal representative of a state of loss of consciousness of the operator or operators to the user device 8, the transmission step E26 being implemented if the determined heart rate is not within the predetermined heart rate range,
a transmission step E27, implemented by the transmission module 29, consisting in or comprising transmitting the signal representative of possible malfunctioning of the image acquisition module 4 to the user device 8, the transmission step E27 being implemented if the determined heart rate is within the predetermined heart rate range.

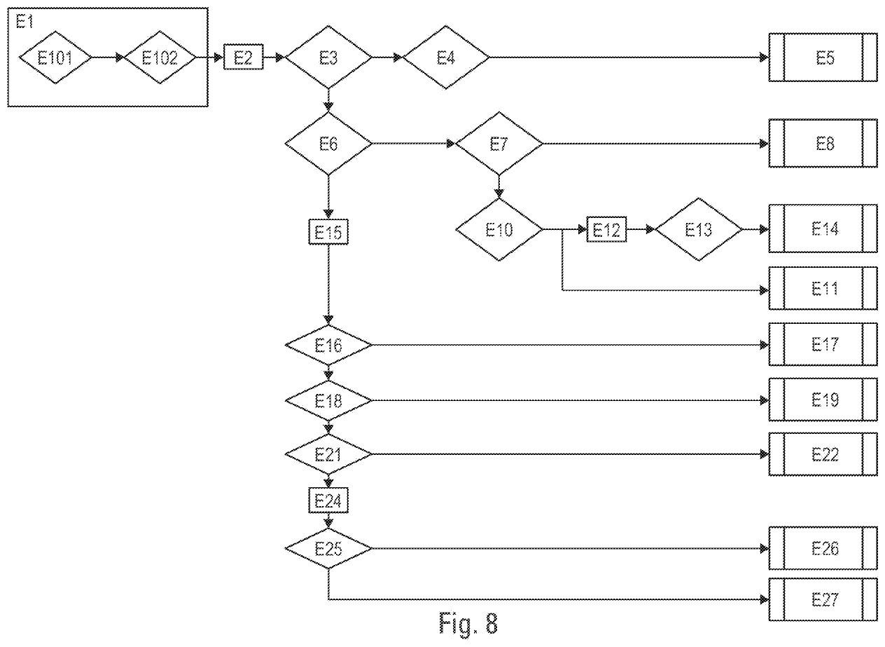

The monitoring method may correspond to a combination of the variants described above, as shown in FIG. 8.

The subject matter disclosed herein can be implemented in software in combination with hardware and/or firmware. For example, the subject matter described herein can be implemented in software executed by a processor or processing unit. In one exemplary implementation, the subject matter described herein can be implemented using a computer readable medium having stored thereon computer executable instructions that when executed by a processor of a computer control the computer to perform steps. Exemplary computer readable mediums suitable for implementing the subject matter described herein include non-transitory devices, such as disk memory devices, chip memory devices, programmable logic devices, and application specific integrated circuits. In addition, a computer readable medium that implements the subject matter described herein can be located on a single device or computing platform or can be distributed across multiple devices or computing platforms.

While at least one example embodiment of the invention(s) is disclosed herein, it should be understood that modifications, substitutions and alternatives may be apparent to one of ordinary skill in the art and can be made without departing from the scope of this disclosure. This disclosure is intended to cover any adaptations or variations of the example embodiment(s). In addition, in this disclosure, the terms “comprise” or “comprising” do not exclude other elements or steps, the terms “a”, “an” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A method for monitoring a state of consciousness of at least one operator in an aircraft cockpit, the method comprising at least one set of steps comprising at least steps as follow, implemented automatically and iteratively:
an image acquisition step, implemented by an image acquisition module, comprising acquiring images of the operator or operators;
a first detection step, implemented by a first detection module, comprising detecting at least one first movement of a body of the operator or operators from the images;
a first determination step, implemented by a first determination module, comprising determining a first movement feature of the body of the operator or operators during at least one first time proportion of a predetermined first time window, the first determination step being implemented if at least one first movement of the body of the operator or operators is detected during at least the first time proportion, the first time proportion of the predetermined first time window corresponding to the ratio between the sum of the times for which the detection is available during the predetermined first time window and the predetermined first time window;
a first transmission step, implemented by a first transmission module, comprising transmitting a signal representative of a state of loss of consciousness of the operator or operators to a user device, the first transmission step being implemented if the first movement feature meets a predetermined first condition.

2. The method according to claim 1, comprising:
a second detection step, implemented by a second detection module, comprising detecting at least one second movement of the body of the operator or operators from the images, the second detection step being implemented:
if no first movement of the body of the operator or operators is detected during the first time proportion; or
if the first movement feature does not meet the predetermined first condition;
a second determination step, implemented by a second determination module, comprising determining a second movement feature of the body of the operator or operators during at least one second time proportion of a second time window, the second determination step being implemented if at least one second movement of the body of the operator or operators is detected during at least the second time proportion;
a second transmission step, implemented by a second transmission module, comprising transmitting a signal representative of a state of loss of consciousness of the operator or operators to the user device, the second transmission step being implemented if the second movement feature meets a predetermined second condition.

3. The method according to claim 2, comprising:
a third transmission step, implemented by a third transmission module, comprising transmitting a signal representative of a possible state of malfunctioning of the movement detection to the user device, the third transmission step being implemented if no second movement of the body of the operator or operators is detected during the second time proportion.

4. The method according to claim 2, wherein the first movement or movements of the body of the operator or operators correspond to at least one blink of at least one eye of the operator or operators, the first movement feature corresponding to a number of blinks of at least one eye, the first movement feature meeting the predetermined first condition if the number of blinks of at least one eye is less than a predetermined number of blinks of at least one eye; and
wherein the second movement or movements of the body of the operator or operators correspond to at least one head direction movement of the operator or operators, the second movement feature corresponding to a head direction movement speed, the second movement feature meeting the predetermined second condition if the head direction movement speed is less than a predetermined head direction movement speed.

5. The method according to claim 2 wherein the first movement or movements of the body of the operator or operators correspond to at least one head direction movement of the operator or operators, the first movement feature corresponding to a head direction movement speed, the first movement feature meeting the predetermined first condition if the head direction movement speed is less than a predetermined head direction movement speed; and
wherein the second movement or movements of the body of the operator or operators correspond to at least one blink of the eye or eyes of the operator or operators, the second movement feature corresponding to a number of blinks of at least one eye, the second movement feature meeting the predetermined second condition if the number of blinks of at least one eye is less than a predetermined number of blinks of at least one eye.

6. The method according to claim 2, comprising:
a third determination step, implemented by a third determination module, comprising determining a head orientation of the operator or operators from the images, the third determination step being implemented if a head direction movement speed is greater than or equal to a predetermined head direction movement speed; and
a fourth transmission step, implemented by a fourth transmission module, comprising transmitting a signal representative of a state of loss of consciousness of the operator to the user device, the second transmission step being implemented if the head orientation is within a predetermined orientation range during at least one third time proportion of the second time window.

7. The method according to claim 2, comprising:
a third detection step, implemented by a third detection module; comprising detecting a shoulder movement of the operator or operators from the images, the third detection step being implemented:
if the second movement feature does not meet the predetermined second condition; or
if the head orientation is greater than or equal to the predetermined orientation during at least the third time proportion;
a fourth determination step, implemented by a fourth determination module, comprising determining a shoulder movement speed of the operator or operators during the third time proportion, the fourth determination step being implemented if at least one shoulder movement of the operator or operators is detected during at least the third time proportion;
a fifth transmission step, implemented by a fifth transmission module, comprising transmitting a signal representative of a state of loss of consciousness of the operator to the user device, the fifth transmission step being implemented if the shoulder movement speed is less than a predetermined shoulder movement speed.

8. The method according to claim 1, comprising an initialization step, implemented by an initialization module, the initialization step comprising at least one first detection substep, implemented by a first detection submodule, comprising detecting an action of the operator or operators on at least one control device on board the aircraft, the set of steps being implemented if no action has been detected during a predetermined first period.

9. The method according to claim 2, comprising:
a fourth detection step, implemented by a fourth detection module, comprising detecting a head direction movement of the operator or operators, the fourth detection step being implemented if no movement of the second body part of the operator or operators has been detected;
a fifth determination step, implemented by a fifth determination module, comprising determining a third head direction movement feature of the operator or operators during at least one fourth time proportion of the second time window, the fifth determination step being implemented if at least one head direction movement of the operator or operators is detected during at least the fourth time proportion;
a sixth transmission step, implemented by a sixth transmission module, comprising transmitting a signal representative of a state of loss of consciousness of the operator to the user device, the sixth transmission step being implemented if the third head direction movement feature meets a predetermined third condition;
a sixth determination step, implemented by a sixth determination module, comprising determining a head direction angle of the operator or operators with respect to an axis of the aircraft from the images, the sixth determination step being implemented if no head direction movement of the operator or operators has been detected;
a seventh transmission step, implemented by a seventh transmission module, comprising transmitting a signal representative of a state of loss of consciousness of the operator to the user device, the seventh transmission step being implemented if the direction angle is less than a predetermined direction angle;
an eighth transmission step, implemented by an eighth transmission module, comprising transmitting a signal representative of possible malfunctioning of the image acquisition module to the user device the eighth transmission step being implemented if the direction angle is greater than or equal to the predetermined direction angle.

10. The method according to claim 8, wherein the initialization step comprises a second detection substep, implemented by a second detection submodule, comprising detecting a pressure exerted by a mass of the operator or operators on a seat on which they are likely to sit, the set of steps being implemented:
firstly if the pressure has been detected during a predetermined second period; or
secondly if an action of the operator or operators has been detected during the predetermined first period and if the pressure has been detected during the predetermined second period;
the method furthermore comprising:
a seventh determination step, implemented by a seventh determination module, comprising determining a distribution of the pressure exerted by the mass of the operator or operators on the seat, the seventh determination step being implemented:
if no second movement of the body of the operator or operators has been detected; or
if the direction angle is greater than or equal to the predetermined direction angle;
a ninth transmission step, implemented by a ninth transmission module, comprising transmitting a signal representative of a state of loss of consciousness of the operator to the user device, the ninth transmission step being implemented if the distribution of the pressure exerted by the mass of the operator or operators on the seat has a difference proportion, in comparison with a reference pressure distribution, less than a predetermined proportion;
a tenth transmission step, implemented by a tenth transmission module, comprising transmitting a signal representative of possible malfunctioning of the image acquisition module to the user device, the tenth transmission step being implemented if the distribution of the pressure exerted by the mass of the operator or operators on the seat has a difference proportion, in comparison with a reference pressure distribution, greater than or equal to the predetermined proportion.

11. The method according to claim 1, comprising:
a fifth detection step, implemented by a fifth detection module, comprising detecting a heartbeat of the operator or operators, the fifth detection step being implemented:
if no second movement of the body of the operator or operators has been detected; or
if the distribution of the pressure exerted by the mass of the operator or operators on the seat has a difference proportion, in comparison with the reference pressure distribution, greater than or equal to the predetermined proportion;
an eighth determination step, implemented by an eighth determination module, comprising determining a heart rate, the eighth determination step being implemented if at least one heartbeat of the operator or operators is detected;
an eleventh transmission step, implemented by an eleventh transmission module, comprising transmitting a signal representative of a state of loss of consciousness of the operator or operators to the user device, the eleventh transmission step being implemented if the determined heart rate is not within a predetermined heart rate range;
a twelfth transmission step, implemented by a twelfth transmission module, comprising transmitting a signal representative of possible malfunctioning of the image acquisition module to the user device, the twelfth transmission step being implemented if the determined heart rate is within the predetermined heart rate range.

12. A system for monitoring a state of consciousness of at least one operator in an aircraft cockpit, the system comprising at least one set of modules comprising at least:
an image acquisition module configured to acquire images of the operator;
a first detection module configured to detect at least one first movement of a body of the operator or operators from the images;
a first determination module configured to determine a first movement feature of the body of the operator or operators during at least one first time proportion of a predetermined first time window, the first determination module being implemented if at least one first movement of the body of the operator or operators is detected during at least the first time proportion, the first time proportion of the predetermined first time window corresponding to a ratio between a sum of times for which the detection is available during the predetermined first time window and the predetermined first time window;
a first transmission module configured s to transmit a signal representative of a state of loss of consciousness of the operator to a user device, the first transmission module being implemented if the first movement feature meets a predetermined first condition.

13. The system according to claim 12, comprising:
a second detection module configured to detect at least one second movement of the body of the operator or operators from the images, the second detection module being implemented:
if no first movement of the body of the operator or operators is detected during the first time proportion; or
if the first movement feature does not meet the predetermined first condition;
a second determination module configured to determine a second movement feature of the body of the operator or operators during at least one second time proportion of a second time window, the second determination module being implemented if at least one second movement of the body of the operator or operators is detected during at least the second time proportion;
a second transmission module configured to transmit a signal representative of a state of loss of consciousness of the operator to the user device, the second transmission module being implemented if the second movement feature meets a predetermined second condition.

14. The system according to claim 12, comprising:
a third transmission module configured to transmit a signal representative of a possible state of malfunctioning of the movement detection to the user device, the third transmission module being implemented if no second movement of the body of the operator or operators is detected during at least the second time proportion.

15. An aircraft, comprising a system according to claim 12 for monitoring a state of consciousness of at least one operator in an aircraft cockpit.
